(19) United States
(12) Patent Application Publication
(10) Pub. No.: US 2022/0344043 A1
(43) Pub. Date: Oct. 27, 2022

(54) Body Area Networks

(71) Applicant: International Business Machines Corporation, Armonk, NY (US)

(72) Inventors: Katsuyuki Sakuma, Fishkill, NY (US); Sarbajit K. Rakshit, Kolkate (IN)

(21) Appl. No.: 17/302,144

(22) Filed: Apr. 26, 2021

(51) Int. Cl. G16H 50/20; G16H 50/30; G16H 10/60; H04W 64/00

(52) U.S. Cl. CPC G16H 50/20; G16H 50/30; G16H 10/60; H04W 64/003; H04W 64/006

(57) ABSTRACT

Identifying physical objects needing sanitization is provided. An object touched by a user is identified by means of a mobile device corresponding to the user. It is determined that the user currently has infectious agent based on retrieved information. The object touched by the user is indicated as needing sanitization.

### BACKGROUND

### 1. Field

The disclosure relates generally to body area networks and more specifically to identifying physical objects potentially contaminated with infectious agents needing sanitization based on data received from mobile electronic devices of body area networks corresponding to users touching the physical objects.

### 2. Description of the Related Art

A body area network, also referred to as a wireless body area network, is a wireless network of electronic devices. Body area network devices may be, for example, surface-mounted on the body, embedded inside the body, or carried in hand, clothes pocket, bag, or the like. The term body area network refers to a network where communication is entirely within, on, and in the immediate proximity of a human body. Typically, a gateway device, such as, for example, a smart phone, makes it is possible to connect the electronic devices of the body area network on the human body to the Internet. The gateway device serves as a data hub, acquiring data and transmitting the data to a server. Other body area network devices may include, for example, smart glasses, smart watches, smart bracelets, smart rings, smart implants such as pacemakers, gaming devices, activity monitors, health monitors, heart rate monitors, and the like.

### SUMMARY

According to one illustrative embodiment, a computer-implemented method for identifying physical objects needing sanitization is provided. An object touched by a user is identified by means of a mobile device corresponding to the user. It is determined that the user currently has an infectious agent based on retrieved information. The object touched by the user is indicated as needing sanitization. According to other illustrative embodiments, a computer system and computer program product for identifying physical objects needing sanitization are provided.

### BRIEF DESCRIPTION OF THE DRAWINGS

FIG. 1 is a pictorial representation of a network of data processing systems in which illustrative embodiments may be implemented;

FIG. 2 is a diagram of a data processing system in which illustrative embodiments may be implemented;

FIG. 3 is a diagram illustrating an example of a contamination management system in accordance with an illustrative embodiment;

FIG. 4 is a diagram illustrating an example of a contamination history record in accordance with an illustrative embodiment;

FIG. 5 is a flowchart illustrating a process for identifying physical objects needing sanitation in accordance with an illustrative embodiment; and

FIG. 6 is a flowchart illustrating a process for sanitizing a physical object contaminated with an infectious agent in accordance with an illustrative embodiment.

### DETAILED DESCRIPTION

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

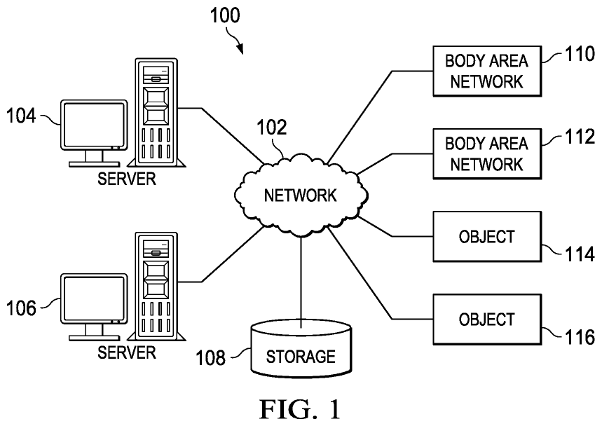

With reference now to the figures, and in particular, with reference to FIGS. 1-3, diagrams of data processing environments are provided in which illustrative embodiments may be implemented. It should be appreciated that FIGS. 1-3 are only meant as examples and are not intended to assert or imply any limitation with regard to the environments in which different embodiments may be implemented. Many modifications to the depicted environments may be made.

FIG. 1 depicts a pictorial representation of a network of data processing systems in which illustrative embodiments may be implemented. Network data processing system 100 is a network of computers, data processing systems, and other devices in which the illustrative embodiments may be implemented. Network data processing system 100 contains network 102, which is the medium used to provide communications links between the computers, data processing systems, and other devices connected together within network data processing system 100. Network 102 may include connections, such as, for example, wire communication links, wireless communication links, fiber optic cables, and the like.

In the depicted example, server 104 and server 106 connect to network 102, along with storage 108. Server 104 and server 106 may be, for example, server computers with high-speed connections to network 102.

Server 104 provides contamination management services to clients. Server 104 provides the contamination management services by identifying physical objects, such as, for example, computers, phones, door handles, and the like, which are touched by users having infectious agents, such as, for example, contagious and transmissible viruses, bacteria, molds, funguses, and the like, that need sanitization. Server 104 determines potential contamination of the physical objects by users who have an infectious agent contacting the physical objects via touching. Further, server 104 provides a warning to potential users of a potentially contaminated object, which was touched by a user having an infectious agent, using, for example, a display system and sound system of the object to display the warning and sound an alert. Furthermore, server 104 may provide the warning using an extended reality system to generate a visualization of the potentially contaminated object touched by the user with the infectious agent. Moreover, server 104 directs an automated sanitization system to clean and sanitize the potentially contaminated object. The automated sanitization system may be a component of the object or may be an independent system, such as, for example, a separate robotic sanitization system.

Server 106 is a medical records server. Server 106 stores a plurality of medical records corresponding to a plurality of individuals. The medical records may include, for example, current test results, current infectious agents (i.e., contagious and transmissible diseases), and the like corresponding to respective individuals. Server 104 can retrieve medical records of particular individuals corresponding to body area network 110 and body area network 112 when these particular individuals physically touch at least one of object 114 or object 116. As a result, server 104 is able to determine whether a particular individual has an infectious agent and whether that particular individual potentially contaminated a particular object with the infectious agent when that particular individual touched that particular object.

Also, it should be noted that server 104 and server 106 may each represent a cluster of servers in one or more data centers. Alternatively, server 104 and server 106 may each represent multiple computing nodes in one or more cloud environments.

Body area network 110, body area network 112, object 114, and object 116 also connect to network 102. Body area network 110, body area network 112, object 114, and object 116 are clients of server 104, which provides the contamination management services. Further, it should be noted that body area network 110, body area network 112, object 114, and object 116 may represent any number of body area networks and objects connected to network 102.

Each of body area network 110 and body area network 112 corresponds to a particular user. A body area network is a short-range wireless network comprised of a set of electronic devices located on, around, and/or in the body of a corresponding user. The set of electronic devices may include at least one of a smart phone, smart glasses, smart watch, smart bracelet, smart ring, smart implant, gaming device, activity monitor, health monitor, heart rate monitor, or the like. In addition, at least one of the set of electronic devices may store a health profile, which indicates whether the user is currently infected with a particular infectious agent. The body area network provides data communication over short distances, limited to ranges of 1-2 meters, for example. The body area network implements useful functionalities and capabilities in configurations that operate at low power and deliver data security.

Each of object 114 and object 116 may represent any type of physical object, such as, for example, a doorknob, a computer keyboard, a landline telephone, an escalator handrailing, or the like, which may be touched by a user corresponding to body area network 110 or body area network 112. Further, each of object 114 and object 116 may include at least one of a transceiver, a processor, a memory, a display system, a sound system, a sanitization system, an extended reality system, or the like.

Storage 108 is a network storage device capable of storing any type of data in a structured format or an unstructured format. In addition, storage 108 may represent a plurality of network storage devices. Further, storage 108 may store identifiers and network addresses for a plurality of medical record servers, identifiers and network addresses for a plurality of body area networks, identifiers for a plurality of users, identifiers and network addresses for a plurality of physical objects, identifiers and network addresses for a plurality of sanitization systems, identifiers and network addresses for a plurality of extended reality systems, and the like.

In addition, it should be noted that network data processing system 100 may include any number of additional servers, body area networks, objects, storage devices, and other devices not shown. Program code located in network data processing system 100 may be stored on a computer-readable storage medium or a set of computer-readable storage media and downloaded to a computer or other data processing device for use. For example, program code may be stored on a computer-readable storage medium on server 104 and downloaded to the set of electronic devices corresponding to body area network 110 over network 102 for use on the set of electronic devices.

In the depicted example, network data processing system 100 may be implemented as a number of different types of communication networks, such as, for example, an internet, an intranet, a wide area network (WAN), a local area network (LAN), a telecommunications network, or any combination thereof. FIG. 1 is intended as an example only, and not as an architectural limitation for the different illustrative embodiments.

As used herein, when used with reference to items, “a number of” means one or more of the items. For example, “a number of different types of communication networks” is one or more different types of communication networks. Similarly, “a set of,” when used with reference to items, means one or more of the items.

Further, the term “at least one of,” when used with a list of items, means different combinations of one or more of the listed items may be used, and only one of each item in the list may be needed. In other words, “at least one of” means any combination of items and number of items may be used from the list, but not all of the items in the list are required. The item may be a particular object, a thing, or a category.

For example, without limitation, “at least one of item A, item B, or item C” may include item A, item A and item B, or item B. This example may also include item A, item B, and item C or item B and item C. Of course, any combinations of these items may be present. In some illustrative examples, “at least one of” may be, for example, without limitation, two of item A; one of item B; and ten of item C; four of item B and seven of item C; or other suitable combinations.

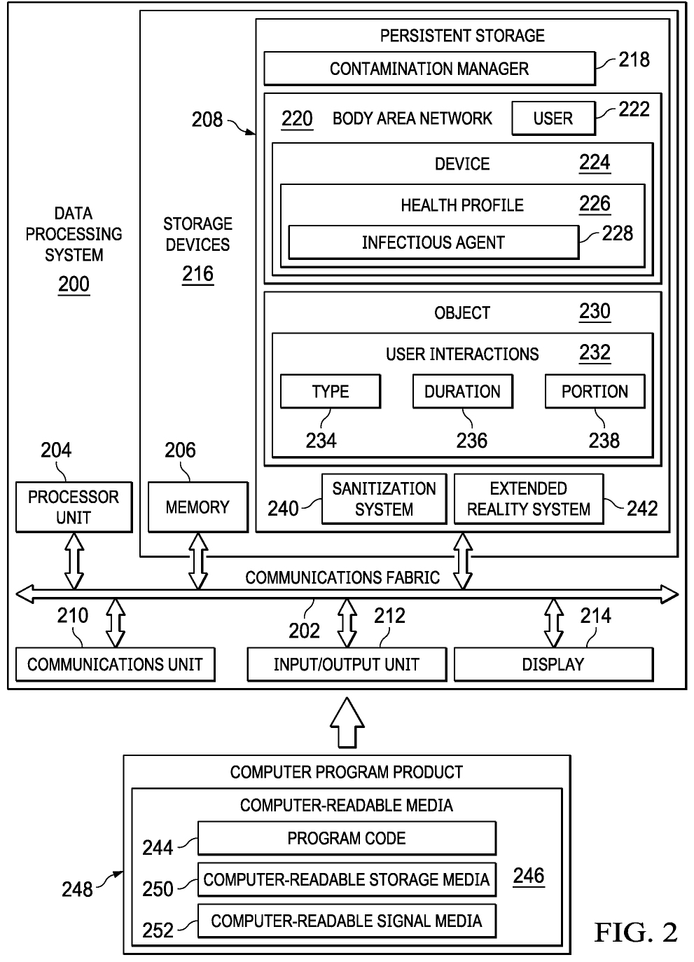

With reference now to FIG. 2, a diagram of a data processing system is depicted in accordance with an illustrative embodiment. Data processing system 200 is an example of a computer, such as server 104 in FIG. 1, in which computer-readable program code or instructions implementing the contamination management processes of illustrative embodiments may be located. In this example, data processing system 200 includes communications fabric 202, which provides communications between processor unit 204, memory 206, persistent storage 208, communications unit 210, input/output (I/O) unit 212, and display 214.

Processor unit 204 serves to execute instructions for software applications and programs that may be loaded into memory 206. Processor unit 204 may be a set of one or more hardware processor devices or may be a multi-core processor, depending on the particular implementation.

Memory 206 and persistent storage 208 are examples of storage devices 216. As used herein, a computer-readable storage device or a computer-readable storage medium is any piece of hardware that is capable of storing information, such as, for example, without limitation, data, computer-readable program code in functional form, and/or other suitable information either on a transient basis or a persistent basis. Further, a computer-readable storage device or a computer-readable storage medium excludes a propagation medium, such as transitory signals. Furthermore, a computer-readable storage device or a computer-readable storage medium may represent a set of computer-readable storage devices or a set of computer-readable storage media. Memory 206, in these examples, may be, for example, a random-access memory (RAM), or any other suitable volatile or non-volatile storage device, such as a flash memory. Persistent storage 208 may take various forms, depending on the particular implementation. For example, persistent storage 208 may contain one or more devices. For example, persistent storage 208 may be a disk drive, a solid-state drive, a rewritable optical disk, a rewritable magnetic tape, or some combination of the above. The media used by persistent storage 208 may be removable. For example, a removable hard drive may be used for persistent storage 208.

In this example, persistent storage 208 stores contamination manager 218. However, it should be noted that even though contamination manager 218 is illustrated as residing in persistent storage 208, in an alternative illustrative embodiment, contamination manager 218 may be a separate component of data processing system 200. For example, contamination manager 218 may be a hardware component coupled to communication fabric 202 or a combination of hardware and software components. Contamination manager 218 controls the process of identifying physical objects potentially contaminated with infectious agents needing sanitization based on data received from mobile electronic devices corresponding to body area networks of users touching the physical objects.

Body area network 220 represents an identifier of a particular body area network, such as, for example, body area network 110 in FIG. 1. Body area network 220 corresponds to user 222. User 222 represents an identifier of a particular user. Device 224 represents identifiers for a set of electronic devices that comprise body area network 220. Device 224 is located on, in, or immediately adjacent to user 222. Device 224 stores health profile 226, which corresponds to user 222. Health profile 226 indicates whether user 222 is currently infected with infectious agent 228. Infectious agent 228 represents any type of contagious and transmissible disease that can be spread by contact with user 222.

Object 230 represents an identifier of a particular physical object and may be, for example, object 114 in FIG. 1. Object 230 may be, for example, a kiosk that individuals may potentially physically contact by touching. User interaction 232 represents a physical contact by user 222 with object 230. User interaction 232 may be, for example, user 222 touching a screen of object 230 (e.g., the kiosk) to make selections. User interaction 232 includes type 234, duration 236, and portion 238. Type 234 represents a type of interaction, such as, for example, touching, grasping, typing, or the like, by user 222 with object 230. Duration 236 represents an amount of time user 222 interacted with object 230. Portion 238 represents which part or parts of object 230 that user 222 contacted.

Device 224 receives data regarding user interaction 232, which includes type 234, duration 236, and portion 238, by user 222 with object 230 via body area network 220. Device 224 sends the data regarding user interaction 232, along with health profile 226 that identifies user 222 as currently having infectious agent 228, to contamination manager 218 for analysis. Based on the data received from device 224, contamination manager 218 determines that user 222 has touched and potentially contaminated object 230 with infectious agent 228.

After determining that object 230 has been touched by user 222 having infectious agent 228, contamination manager 218 directs sanitization system 240 to clean and disinfect object 230 for infectious agent 228. Sanitization system 240 is an independent automated system, such as, for example, a separate robotic sanitization system. Alternatively, sanitization system 240 is a component of object 230. Further, contamination manager 218 may direct extended reality system 242 to generate a visualization of infectious agent 228 on object 230 prior to sanitization system 240 completing the sanitization process. Extended reality system 242 may be a component of object 230 or may be an independent system, such as, for example, an extended reality headset worn by a user. Extended reality system 242 includes at least one of a virtual reality system, an augmented reality system, or a mixed reality system.

As a result, data processing system 200 operates as a special purpose computer system in which contamination manager 218 in data processing system 200 enables detection of potentially contaminated objects, which were touched by users having infectious agents, needing sanitization. In particular, contamination manager 218 transforms data processing system 200 into a special purpose computer system as compared to currently available general computer systems that do not have contamination manager 218.

Communications unit 210, in this example, provides for communication with other computers, data processing systems, and devices via a network, such as network 102 in FIG. 1. Communications unit 210 may provide communications through the use of both physical and wireless communications links. The physical communications link may utilize, for example, a wire, cable, universal serial bus, or any other physical technology to establish a physical communications link for data processing system 200. The wireless communications link may utilize, for example, shortwave, high frequency, ultrahigh frequency, microwave, wireless fidelity (Wi-Fi), Bluetooth® technology, global system for mobile communications (GSM), code division multiple access (CDMA), second-generation (2G), third-generation (3G), fourth-generation (4G), 4G Long Term Evolution (LTE), LTE Advanced, fifth-generation (5G), or any other wireless communication technology or standard to establish a wireless communications link for data processing system 200.

Input/output unit 212 allows for the input and output of data with other devices that may be connected to data processing system 200. For example, input/output unit 212 may provide a connection for user input through a keypad, a keyboard, a mouse, a microphone, and/or some other suitable input device. Display 214 provides a mechanism to display information to a user and may include touch screen capabilities to allow the user to make on-screen selections through user interfaces or input data, for example.

Instructions for the operating system, applications, and/or programs may be located in storage devices 216, which are in communication with processor unit 204 through communications fabric 202. In this illustrative example, the instructions are in a functional form on persistent storage 208. These instructions may be loaded into memory 206 for running by processor unit 204. The processes of the different embodiments may be performed by processor unit 204 using computer-implemented instructions, which may be located in a memory, such as memory 206. These program instructions are referred to as program code, computer usable program code, or computer-readable program code that may be read and run by a processor in processor unit 204. The program instructions, in the different embodiments, may be embodied on different physical computer-readable storage devices, such as memory 206 or persistent storage 208.

Program code 244 is located in a functional form on computer-readable media 246 that is selectively removable and may be loaded onto or transferred to data processing system 200 for running by processor unit 204. Program code 244 and computer-readable media 246 form computer program product 248. In one example, computer-readable media 246 may be computer-readable storage media 250 or computer-readable signal media 252.

In these illustrative examples, computer-readable storage media 250 is a physical or tangible storage device used to store program code 244 rather than a medium that propagates or transmits program code 244. Computer-readable storage media 250 may include, for example, an optical or magnetic disc that is inserted or placed into a drive or other device that is part of persistent storage 208 for transfer onto a storage device, such as a hard drive, that is part of persistent storage 208. Computer-readable storage media 250 also may take the form of a persistent storage, such as a hard drive, a thumb drive, or a flash memory that is connected to data processing system 200.

Alternatively, program code 244 may be transferred to data processing system 200 using computer-readable signal media 252. Computer-readable signal media 252 may be, for example, a propagated data signal containing program code 244. For example, computer-readable signal media 252 may be an electromagnetic signal, an optical signal, or any other suitable type of signal. These signals may be transmitted over communication links, such as wireless communication links, an optical fiber cable, a coaxial cable, a wire, or any other suitable type of communications link.

Further, as used herein, “computer-readable media 246” can be singular or plural. For example, program code 244 can be located in computer-readable media 246 in the form of a single storage device or system. In another example, program code 244 can be located in computer-readable media 246 that is distributed in multiple data processing systems. In other words, some instructions in program code 244 can be located in one data processing system while other instructions in program code 244 can be located in one or more other data processing systems. For example, a portion of program code 244 can be located in computer-readable media 246 in a server computer while another portion of program code 244 can be located in computer-readable media 246 located in a set of client computers.

The different components illustrated for data processing system 200 are not meant to provide architectural limitations to the manner in which different embodiments can be implemented. In some illustrative examples, one or more of the components may be incorporated in or otherwise form a portion of, another component. For example, memory 206, or portions thereof, may be incorporated in processor unit 204 in some illustrative examples. The different illustrative embodiments can be implemented in a data processing system including components in addition to or in place of those illustrated for data processing system 200. Other components shown in FIG. 2 can be varied from the illustrative examples shown. The different embodiments can be implemented using any hardware device or system capable of running program code 244.

In another example, a bus system may be used to implement communications fabric 202 and may be comprised of one or more buses, such as a system bus or an input/output bus. Of course, the bus system may be implemented using any suitable type of architecture that provides for a transfer of data between different components or devices attached to the bus system.

Contamination can be transmitted via human contact. For example, when a user physically touches or grips an object, the user can potentially contaminate the object or the object can potentially contaminate the user. It should be noted that if a user who currently has an infectious agent can potentially contaminate an object via physical touch, then that object is considered contaminated by that infectious agent. As a result, sanitization of contaminated objects is needed where human interaction via physical contact with these contaminated objects is possible.

Illustrative embodiments identify potentially contaminated objects needing sanitization before reuse of these potentially contaminated objects to stop propagation of the contamination. By analyzing data received from a set of body area network devices corresponding to a user, illustrative embodiments can identify potentially contaminated objects interacted with (e.g., touched, gripped, held, typed on, and the like) by a user who may be infected with an infectious agent (i.e., a contagious and transmissible disease or the like). Thus, illustrative embodiments can identify the objects needing sanitization for the infectious agent. The infectious agent may be, for example, a virus, bacteria, mold, fungus, or the like. The object may be, for example, an inanimate object, such as, for example, a doorknob or handle, handrail, arm rest, grocery chart, computer, phone, kiosk, steering wheel, or the like. The object may also be an animate object, such as, for example, a human or an animal.

Illustrative embodiments track body area network signals and identify whether an object is potentially contaminated from a particular source. If illustrative embodiments identify that an object is potentially contaminated with an infectious agent due to touching by a person who has the infectious agent, then illustrative embodiments may utilize an extended reality device, such as, for example, a virtual reality, augmented reality, or mixed reality device, to highlight the object or that portion of the object that is potentially contaminated to warn others in the surrounding area regarding the potential contamination.

Further, illustrative embodiments track and record whether appropriate sanitization was applied to the object touched by the user with the infectious agent. For example, illustrative embodiments track the types of objects to be sanitized and the types of infectious agents potentially on the objects and direct a sanitization system to perform appropriate sanitization of a particular object based on the type of that particular object and the type of the infectious agent. Furthermore, illustrative embodiments remove contamination status of the object after the sanitization system completes sanitation procedures for the object.

Each user has a set of mobile electronic devices located on, in, and/or adjacent to the body of a respective user that generates a body area network corresponding to the user. Illustrative embodiments uniquely identify the user using the set of mobile electronic devices corresponding to the user.

A body area network device corresponding to the user can store a health or medical profile, which may include, for example, current infectious agents (i.e., current contagious and transmissible diseases) of the user. Illustrative embodiments can retrieve the health profile of the user from the body area network device. Illustrative embodiments may also retrieve medical records, such as, medical conditions, test results, current infections, and the like, corresponding to the user from one or more remote medical record servers.

Also, the body area network of the user may receive signals from other body area networks corresponding to other users within a defined area. The signals identify which objects in the defined area have been touched by the other users. The body area network devices transmit these signals to a server for analysis. For example, when a user physically contacts by touching an object, the body area network device starts sending signals through the body of the user and a receiving unit of the body area network corresponding to the user receives the signals. In other words, electronic signals of the body area network pass through the body of the user and move toward the object that the user is touching. The receiving unit receives the signals from the body area network device via the body area network corresponding to the user. The receiving unit identifies the type of user interaction with the object (e.g., touch, grip, typing, or the like), duration of the user interaction, location of the user interaction on the object, and the like. The body area network device transmits this user interaction data to the server via a network.

The server analyzes the user interaction data to identify, for example, who in particular interacted with the object via touch, which object was interacted with by the user, which portion of the object was interacted with by the user, the type of the user interaction, the duration of the user interaction, and the like. Further, the server identifies whether the user has a current infectious agent based on at least one of information in the health profile of the user retrieved from the body area network device or information in medical records corresponding to the user retrieved from one or more remote medical record servers. If the server determines that the user has a current infectious agent based on the retrieved information corresponding to the user, then the server identifies the appropriate sanitization procedures to be performed by a sanitization system based on the object, the user interaction, and the type of infectious agent. Afterward, the server directs the sanitization system to perform the appropriate sanitization of the object. The sanitization system may be, for example, an independent automated sanitization system, such as a separate robotic sanitization system. Alternatively, the object, itself, may contain the sanitization system.

Illustrative embodiments also track any cross-interaction with the object, such as, for example, a doctor interacting with a contaminated patient. In this example, the body area network device of the doctor identifies the contaminant transferred to the doctor and sends that information to the server for analysis. The server identifies the object (e.g., the doctor) that potentially became contaminated and needing sanitization before the object can potentially contaminate other objects (e.g., other patients). Further, illustrative embodiments can generate an extended reality visualization using an extended reality system to show which object or which portion of the object is potentially contaminated and needing sanitization. After appropriate sanitization is applied to the potentially contaminated object, illustrative embodiments delete historical data regarding the potential transmission of the infectious agent by the object and show the object as free from the infectious agent.

Thus, illustrative embodiments provide one or more technical solutions that overcome a technical problem with identifying contaminated objects needing sanitization based on data received from body area networks. As a result, these one or more technical solutions provide a technical effect and practical application in the field of body area networks.

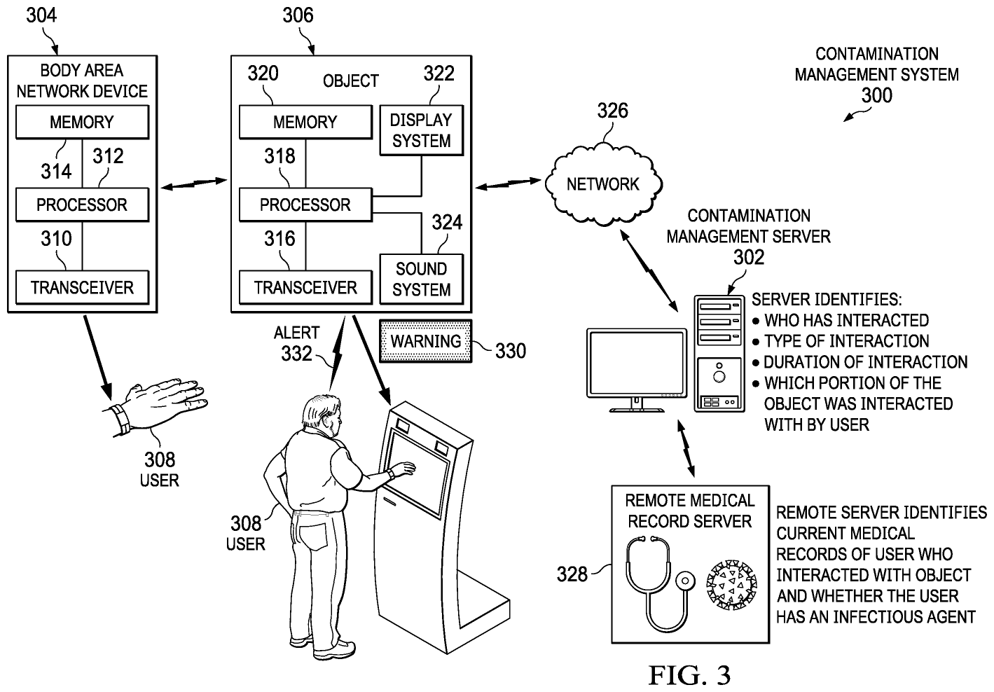

With reference now to FIG. 3, a diagram illustrating an example of a contamination management system is depicted in accordance with an illustrative embodiment. Contamination management system 300 may be implemented in a network of data processing systems, such as network data processing system 100 in FIG. 1. Contamination management system 300 is a system of components for identifying potentially contaminated physical objects, which were touched by users who have infectious agents, needing sanitization based on data received from mobile electronic devices corresponding to body area networks of the users who touched the physical objects.

In this example, contamination management system 300 includes contamination management server 302, body area network device 304, object 306, and user 308. Contamination management server 302 may be, for example, server 104 in FIG. 1 or data processing system 200 in FIG. 2. Body area network device 304 may be, for example, device 224 of body area network 220 in FIG. 2. Object 306 may be, for example, object 114 in FIG. 1 or object 230 in FIG. 2. User 308 may be, for example, user 222 in FIG. 2.

In this example, body area network device 304 is a smart bracelet or smart watch, with integrated body area network functionality, worn by user 308. Body area network device 304 includes transceiver 310, processor 312, and memory 314. Object 306 in this example is a kiosk touched by user 308. Object 306 has integrated body area network functionality and includes transceiver 316, processor 318, memory 320, display system 322, and sound system 324. In an alternative illustrative embodiment, object 306 may not include display system 322 and sound system 324. In addition, it should be noted that object 306 utilizes a set of sensors having the capability of detecting which portion of object 306 that user 308 touched. Object 306 transmits data regarding which portion of object 306 that user 308 touched to body area network device 304 via the body area network corresponding to user 308.

Body area network device 304 utilizes transceiver 310 to send data regarding physical contact by user 308 with object 306, such as, for example, user interaction 232 corresponding to object 230 in FIG. 2, to contamination management server 302 for analysis via network 326. Network 326 may be, for example, network 102 in FIG. 1. Body area network device 304 sends the data regarding the user interaction when user 308 physically contacts object 306 via touch.

Upon receiving the user interaction data from body area network device 304, contamination management server 302 identifies who exactly (i.e., user 308) interacted with object 306, the type of the user interaction, the duration of the user interaction, which portion of object 306 was interacted with by user 308, and the like. Body area network device 304 knows which portion of object 306 the user 308 touched based on the data received from object 306 obtained from the set of sensors. In addition, contamination management server 302 retrieves a set of medical records corresponding to user 308 from remote medical records server 328 to determine whether user 308 currently has an infectious agent.

If contamination management server 302 determines that user 308 is currently infected with an infectious agent based on information in the set of medical records corresponding to user 308 retrieved from remote medical records server 328 and that user 308 touched object 306 based on the received user interaction data received from body area network device 304, then contamination management server 302 sends instructions to object 306 via transceiver 316 to display warning 330 on display system 322 and output alert 332 on sound system 324 to inform potential users that object 306 is potentially contaminated with the infectious agent. Furthermore, contamination management server 302 may direct a sanitization system, such as, for example, sanitization system 240 in FIG. 2, to clean and disinfect object 306.

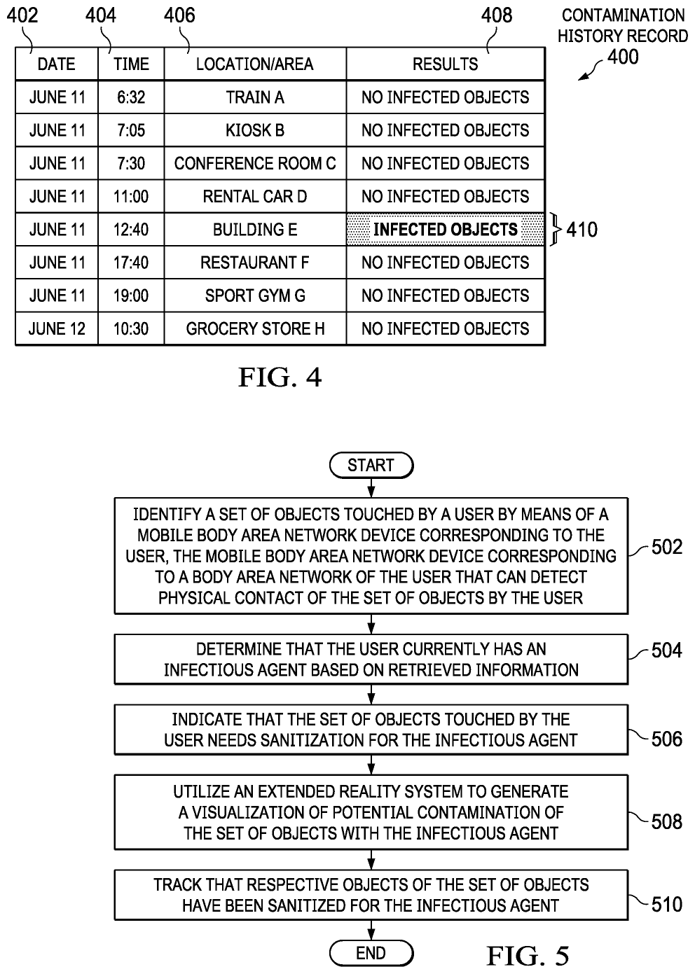

With reference now to FIG. 4, a diagram illustrating an example of a contamination history record is depicted in accordance with an illustrative embodiment. Contamination history record 400 may be implemented in computer, such as, for example, server 104 in FIG. 1, data processing system 200 in FIG. 2, or contamination management server 302 in FIG. 3.

In this example, contamination history record 400 includes date 402, time 404, location/area 406, and results 408. However, it should be noted that contamination history record 400 is intended as an example only and not as a limitation on illustrative embodiments. In other words, contamination history record 400 may include more or less information than illustrated. For example, contamination history record 400 may also include other information such as type of infection, appropriate sanitization procedures for that type of infection, when the object was sanitized for the infection, and the like.

Results 408 indicates whether any objects in a particular location/area identified in location/area 406 are potentially infected with an infectious agent. In this example, row 410 indicates that potentially infected objects were detected in Building E on June 11 at 12:40.

With reference now to FIG. 5, a flowchart illustrating a process for identifying physical objects needing sanitation is shown in accordance with an illustrative embodiment. The process shown in FIG. 5 may be implemented in a computer, such as, for example, server 104 in FIG. 1, data processing system 200 in FIG. 2, or contamination management server 302 in FIG. 3.

The process begins when the computer identifies a set of objects touched by a user by means of a mobile body area network device corresponding to the user (step 502). The mobile body area network device corresponds to a body area network of the user that can detect physical contact between the user and the set of objects. In addition, the computer determines that the user currently has an infectious agent (step 504). Further, the computer indicates that the set of objects touched by the user needs sanitization for the infectious agent (step 506).

Furthermore, the computer utilizes an extended reality system to generate a visualization of potential contamination of the set of objects with the infectious agent (step 508). The computer also provides the user with a preferred sanitization procedure for each respective object of the set of objects. Moreover, the computer tracks that respective objects of the set of objects have been sanitized for the infectious agent (step 510). Thereafter, the process terminates.

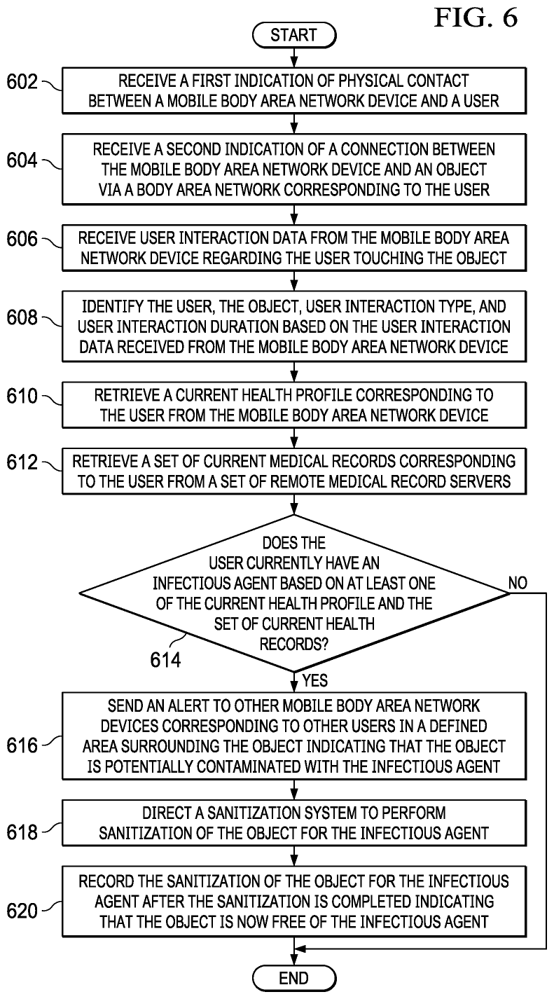

With reference now to FIG. 6, a flowchart illustrating a process for sanitizing a physical object contaminated with an infectious agent is shown in accordance with an illustrative embodiment. The process shown in FIG. 6 may be implemented in a computer, such as, for example, server 104 in FIG. 1, data processing system 200 in FIG. 2, or contamination management server 302 in FIG. 3.

The process begins when the computer receives a first indication of physical contact between a mobile body area network device and a user (step 602). The computer receives a second indication of a connection between the mobile body area network device and an object via a body area network corresponding to the user (step 604). The computer receives user interaction data from the mobile body area network device regarding the user touching the object (step 606).

Afterward, the computer identifies the user, the object, user interaction type, and user interaction duration based on the user interaction data received from the mobile body area network device (step 608). In addition, the computer retrieves a current health profile corresponding to the user from the mobile body area network device (step 610). Further, the computer retrieves a set of current medical records corresponding to the user from a set of remote medical record servers (step 612).

The computer makes a determination as to whether the user currently has an infectious agent based on at least one of the current health profile and the set of current medical records (step 614). If the computer determines that the user does not currently have an infectious agent based on at least one of the current health profile and the set of current health records, no output of step 614, then the process terminates thereafter. If the computer determines that the user does currently have an infectious agent based on at least one of the current health profile and the set of current health records, yes output of step 614, then the computer sends an alert to other mobile body area network devices corresponding to other users in a defined area surrounding the object indicating that the object is potentially contaminated with the infectious agent (step 616).

Furthermore, the computer directs a sanitization system to perform appropriate sanitization of the object for the infectious agent (step 618). Afterward, the computer records the sanitization of the object for the infectious agent indicating that the object is now free of the infectious agent (step 620). Thereafter, the process terminates.

Thus, illustrative embodiments of the present invention provide a computer-implemented method, computer system, and computer program product for identifying physical objects potentially contaminated with infectious agents needing sanitization based on data received from mobile electronic devices corresponding to body area networks of users touching the physical objects. The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for identifying physical objects needing sanitization, the method comprising:
identifying an object touched by a user by means of a mobile device corresponding to the user;
determining that the user currently has an infectious agent based on retrieved information; and
indicating that the object touched by the user needs sanitization.

2. The method of claim 1 further comprising:
utilizing an extended reality system to generate a visualization of potential contamination of the object with the infectious agent.

3. The method of claim 1 further comprising:
sending an alert to other mobile devices corresponding to other users in a defined area surrounding the object indicating that the object is potentially contaminated with the infectious agent, wherein potential contamination of the object is determined by the user contacting the object.

4. The method of claim 1 further comprising:
directing a sanitization system to perform sanitization of the object for the infectious agent.

5. The method of claim 1 further comprising:
tracking the sanitization of the object for the infectious agent; and
recording the sanitization of the object for the infectious agent after the sanitization is completed indicating that the object is free of the infectious agent.

6. The method of claim 1 further comprising:
retrieving a health profile corresponding to the user from the mobile device;
retrieving a medical record corresponding to the user from a medical record server; and
determining whether the user currently has the infectious agent based on at least one of the health profile and the medical record.

7. The method of claim 1 further comprising:
receiving user interaction data from the mobile device regarding the user touching the object; and
identifying the user, the object, user interaction type, and user interaction duration based on the user interaction data received from the mobile device.

8. The method of claim 1 further comprising:
receiving a first indication of contact between the mobile device and the user; and
receiving a second indication of a connection between the mobile device and the object via a body area network corresponding to the user.

9. The method of claim 8, wherein the mobile device is a mobile body area network device corresponding to the body area network of the user, the body area network detecting physical contact between the user and the object.

10. The method of claim 1 further comprising:
providing the user with a preferred sanitization procedure for the object.

11. The method of claim 1 further comprising:
using a display system and a sound system of the object to display a warning and sound an alert to inform potential users that the object is potentially contaminated with the infectious agent.

12. A computer system for identifying physical objects needing sanitization, the computer system comprising:
a bus system;
a storage device connected to the bus system, wherein the storage device stores program instructions; and
a processor connected to the bus system, wherein the processor executes the program instructions to:
identify an object touched by a user by means of a mobile device corresponding to the user;
determine that the user currently has an infectious agent based on retrieved information; and
indicate that the object touched by the user needs sanitization.

13. The computer system of claim 12, wherein the processor further executes the program instructions to:
utilize an extended reality system to generate a visualization of potential contamination of the object with the infectious agent.

14. The computer system of claim 12, wherein the processor further executes the program instructions to:
send an alert to other mobile devices corresponding to other users in a defined area surrounding the object indicating that the object is potentially contaminated with the infectious agent, wherein potential contamination of the object is determined by the user contacting the object.

15. The computer system of claim 12, wherein the processor further executes the program instructions to:
direct a sanitization system to perform sanitization of the object for the infectious agent.

16. A computer program product for identifying physical objects needing sanitization, the computer program product comprising a computer-readable storage medium having program instructions embodied therewith, the program instructions executable by a computer to cause the computer to perform a method of:
identifying an object touched by a user by means of a mobile device corresponding to the user;
determining that the user currently has an infectious agent based on retrieved information; and
indicating that the object touched by the user needs sanitization.

17. The computer program product of claim 16 further comprising:
utilizing an extended reality system to generate a visualization of potential contamination of the object with the infectious agent.

18. The computer program product of claim 16 further comprising:
sending an alert to other mobile devices corresponding to other users in a defined area surrounding the object indicating that the object is potentially contaminated with the infectious agent, wherein potential contamination of the object is determined by the user contacting the object.

19. The computer program product of claim 16 further comprising:
directing a sanitization system to perform sanitization of the object for the infectious agent.

20. The computer program product of claim 16 further comprising:
tracking the sanitization of the object for the infectious agent; and
recording the sanitization of the object for the infectious agent after the sanitization is completed indicating that the object is free of the infectious agent.
